(19) United States
(12) Patent Application Publication
(10) Pub. No.: US 2026/0240459 A1
(43) Pub. Date: Aug. 20, 2026

(54) Multispectral Imaging Device for Non-Invasive Disease Detection

(71) Applicant: Alice Yang, Troy, MI (US)

(72) Inventor: Alice Yang, Troy, MI (US)

(21) Appl. No.: 19/055,062

(22) Filed: Feb. 17, 2025

(51) Int. Cl. A61B 5/1455; A61B 5/00; A61B 5/145; G06T 7/00

(52) U.S. Cl. CPC A61B 5/1455; A61B 5/14532; A61B 5/14546; A61B 5/6898; G06T 7/0012; G06T 2207/10036; G06T 2207/30041; G06T 2207/30088

(57) ABSTRACT

This invention relates to a low-cost portable multispectral detection method and device for early disease diagnosis. The device is based on a CMOS multispectral sensor chip and uses natural light or halogen lamps as the light source to non-invasively detect physiological parameters from the eyes, oral cavity, and/or skin of patients. The device captures reflected light data of different wavelengths using the multispectral chip and processes the data to generate image information, subsequently identifying the corresponding test areas. By processing the initial spectral information, the device provides accurate spectral data analysis to determine physiological parameters such as glucose etc., enabling early diagnosis of diseases such as diabetes, etc. It features real-time data analysis and wireless data transmission, offering a convenient solution for large-scale health monitoring and telemedicine.

### FIELD OF THE INVENTION

This invention relates to the field of medical imaging devices and diagnostic technologies, specifically to a portable multispectral imaging device based on a CMOS spectral sensor chip that supports multispectral acquisition and computational reconstruction. In the chip, each pixel is equipped with a corresponding microlens, color filter, and photodiode. Each spectrum has its corresponding filter and photodiode, and they are directly matched. A focus-adjustable, filter-free lens can be used for the spectral sensor chip to detect light. If a lens is not used, it can work in certain specific scenarios, such as when the multispectral chip is positioned close to the eyes, oral cavity, and/or skin.

The device uses natural light or halogen lamps as light sources to non-invasively detect glucose, bilirubin, oxyhemoglobin (HbO2), and deoxyhemoglobin (Hb) concentrations in the patient's eyes, oral cavity, and/or skin, enabling early diagnosis of diseases including diabetes, liver cancer, pancreatic cancer, and lymphoma. The chip has high sensitivity and can work with either natural light or halogen lamps, as both include the full spectrum of natural light. When natural light is sufficient, it can be used, and when it is insufficient, halogen lamps can be used. The whites of the eyes are relatively clean, making detection in this area the most accurate. In contrast, the skin or oral cavity is harder to clean, resulting in slightly lower accuracy.

The algorithms in this invention are as follows:

Gather spectral information from multiple channels to form a spectral information sample. Compare the spectral information sample with samples in the spectral database to identify target samples that are identical or similar to the spectral information sample.

Use the target sample's corresponding physiological parameters as the target physiological parameters. Fit spectral information from multiple channels to form a sample spectral curve. Compare the sample spectral curve with sample spectral curves in the spectral database to identify target spectral curves that are identical or similar to the sample spectral curve.

Based on spectral information from multiple channels, generate a sample spectral image. Compare the sample spectral image with sample spectral images in the spectral database to identify target spectral images that are identical or similar to the sample spectral image.

Input the spectral information sample into a spectral model, which is a neural network model, for further analysis.

Spectra are often regarded as the “fingerprints” that distinguish substances, and multispectral imaging devices serve as the “smart glasses” that help us clearly see these “fingerprints”.

The device is suitable for use in hospitals, clinics, home care, and telemedicine, demonstrating wide application potential.

In addition to personalized medical applications, the device can also be integrated into health monitoring systems to enable remote data transmission and large-scale health management services. The development and promotion of this device will significantly improve the efficiency of early screening and disease management, advancing the field of precision medicine.

### BACKGROUND OF THE INVENTION

Diabetes is a globally prevalent metabolic disease, with long-term high blood glucose levels leading to serious complications such as cardiovascular disease, neuropathy, and retinopathy. According to the World Health Organization (WHO), the number of diabetes patients worldwide continues to rise, especially in developing countries.

Diabetes is a chronic condition where blood glucose levels cannot be regulated due to insufficient insulin production or resistance. Type 1 diabetes results from an autoimmune reaction stopping insulin production, while type 2 involves insulin resistance. About 5-10% of diabetes patients have type 1, with type 2 accounting for over 90%. Elevated blood glucose causes complications like cardiovascular diseases, and kidney and eye damage. In the U.S., over 37 million adults have diabetes, with 463 million worldwide. Over 96 million U.S. adults have pre-diabetes.

Type 1 diabetes often has noticeable symptoms like fatigue and thirst, while type 2 may go unnoticed longer, with many undiagnosed. Early detection and monitoring are crucial. Diabetes management includes lifestyle changes, insulin, and medication. Regular blood glucose monitoring is vital to avoid complications, but current methods like finger pricking are uncomfortable and costly.

Non-invasive glucose monitoring has been explored using various technologies like infrared spectroscopy, bioimpedance, and photoplethysmography (PPG) via smartphones. These approaches require less invasive procedures but have struggled with reliability and accuracy. For widespread acceptance, these methods must match the accuracy of conventional blood glucose meters, with a mean absolute relative difference below 20%. Thus, there is a need for improved non-invasive glucose monitoring systems.

Current blood glucose monitoring methods are mostly invasive, such as blood glucose testing via finger-prick sampling. Although continuous glucose monitoring (CGM) devices have partially addressed this issue, they remain expensive and still require frequent sensor replacements.

Bilirubin is a byproduct of red blood cell metabolism, and abnormal elevations can lead to jaundice, commonly associated with liver diseases (e.g., liver cancer, cirrhosis) and certain cancers (e.g., pancreatic cancer). Abnormal bilirubin levels not only affect the patient's appearance but also reflect metabolic dysfunction in the liver. Traditional methods of detecting bilirubin rely on blood testing and biochemical analysis, which are invasive, complex, and time-consuming.

Oxyhemoglobin (HbO2) and deoxyhemoglobin (Hb) play a crucial role in oxygen transport in the body, and their concentration changes can effectively reflect the oxygenation and metabolic status of tissues. Many cancers (such as pancreatic cancer, lung cancer, and lymphoma) in their early stages alter the blood flow and oxygen consumption in local tissues, resulting in an imbalance between oxyhemoglobin and deoxyhemoglobin. Monitoring the changes in hemoglobin composition can provide early indications of disease progression.

Multispectral imaging technology can capture spectral information across multiple bands, offering greater accuracy in identifying biochemical changes within tissues compared to traditional imaging methods. As such, multispectral imaging is becoming a valuable emerging technology in medical imaging, particularly for the early detection of diseases like cancer and diabetes.

Principles and applications of multispectral detection are:

Glucose Detection: Glucose molecules in skin tissue have specific spectral characteristics in the near-infrared region with Glucose-sensitive wavelength range: 940-1050 nm. By analyzing spectral data across different wavelengths, glucose concentrations can be accurately calculated. This technology overcomes the limitations of traditional finger-prick glucose testing, avoiding invasive procedures while enabling more frequent monitoring. For diabetes patients, continuous glucose monitoring through this device not only prevents risks of hyperglycemia or hypoglycemia but also helps doctors adjust treatment plans in real time, preventing diabetes-related complications.

Bilirubin Detection: Bilirubin has distinct absorption characteristics in the blue light region with Bilirubin sensitive wavelength range: 450-480 nm. Through multispectral imaging, the concentration of bilirubin in the skin can be quantitatively detected, providing detailed information about liver function. The device can capture abnormalities in bilirubin metabolism during early disease stages, such as liver damage or bile duct obstruction. For liver cancer or pancreatic cancer patients, early detection of changes in bilirubin levels facilitates timely intervention and improves patient prognosis.

Oxyhemoglobin (HbO2) and Deoxyhemoglobin (Hb) Detection: Oxyhemoglobin and deoxyhemoglobin exhibit different absorption characteristics in the red and near-infrared regions with Oxyhemoglobin (HbO2) sensitive wavelength range: 540-580 nm (green to yellow region) and 940 nm (near-infrared), and Deoxyhemoglobin (Hb) sensitive wavelength range: 520-550 nm (green region) and 900-940 nm (near-infrared).

Through multispectral imaging, the oxygenation status of local tissues can be detected. This technique is particularly important for early cancer screening, as tumor growth often leads to abnormal blood supply and oxygen utilization in surrounding tissues, altering the HbO2 to Hb ratio. By detecting these changes, the device can provide valuable early diagnostic information about the histological characteristics of cancer, aiding in early intervention.

Applications for Diabetes: Diabetes not only affects blood glucose levels but also causes systemic metabolic changes. By monitoring glucose and HbO2/Hb concentrations, the device can assess the metabolic status of diabetes patients, evaluate the risk of cardiovascular and neurological complications, and assist doctors in personalizing treatment plans. The monitoring function of the device enables patients to manage their condition themselves, greatly improving their quality of life.

Liver Cancer Application: Liver cancer (hepatocellular carcinoma) is closely associated with metabolic changes in liver function, often beginning with disruptions in bile production and blood filtration. Tumors in the liver can cause significant alterations in blood flow within the hepatic portal system, leading to reduced oxygenation and increased bilirubin levels as liver function deteriorates. Elevated bilirubin, resulting in jaundice, is a hallmark sign of liver dysfunction, particularly in cases of liver cancer.

The proposed device, through multispectral imaging, can detect these changes by measuring bilirubin concentrations in the skin, providing early indications of liver damage. Additionally, the device can capture changes in the oxygenation status of liver tissues by analyzing the levels of HbO2 and Hb, which are often impacted by the tumor's presence. Liver tumors disrupt the normal flow of oxygen-rich blood, leading to hypoxia in the surrounding areas. Detecting these early-stage metabolic anomalies offers a crucial window for early diagnosis, potentially allowing for more effective intervention before the cancer advances.

Furthermore, since liver cancer is often preceded by liver diseases like cirrhosis or hepatitis, the device can also be used as a preventive screening tool in high-risk populations. By monitoring bilirubin levels and tissue oxygenation over time, healthcare providers can detect early liver dysfunction, providing an opportunity for early treatment before cancer fully develops.

Pancreatic Cancer Application: In the early stages of pancreatic cancer, tumor growth can disrupt the blood supply and bile duct function in tissues surrounding the pancreas. Pancreatic tumors often cause localized inflammation and necrosis, resulting in altered tissue oxygenation and disrupted bilirubin metabolism due to bile duct obstruction. By using the device to non-invasively measure bilirubin levels in the skin, early signs of bile duct dysfunction can be detected, which is often a precursor to pancreatic disease. Furthermore, the device can detect imbalances in the levels of oxyhemoglobin (HbO2) and deoxyhemoglobin (Hb) in the surrounding tissues, reflecting localized metabolic changes and reduced oxygenation.

Such metabolic disruptions are common in the early stages of pancreatic cancer, where the tumor's influence on blood flow and oxygen consumption can lead to hypoxia (oxygen deficiency) in adjacent tissues. Multispectral imaging technology, by detecting these subtle changes in skin and tissue oxygenation, offers a promising approach for the early identification of pancreatic cancer. This non-invasive method not only provides a means for early diagnosis but also serves as a tool for ongoing monitoring of disease progression and treatment efficacy.

Lymphoma Application: Lymphoma, a cancer that originates in the lymphatic system, can cause abnormalities in both blood flow and oxygen consumption within the lymph nodes and surrounding tissues. As lymphoma progresses, the tumor can interfere with the lymphatic drainage and immune response, leading to changes in tissue metabolism. Multispectral imaging technology is particularly useful in detecting the altered metabolic state in affected lymph nodes by measuring changes in the concentration of oxyhemoglobin (HbO2) and deoxyhemoglobin (Hb) in these regions.

By capturing these changes, the device can reveal early signs of lymphoma, which may otherwise go unnoticed in the absence of more overt clinical symptoms. The non-invasive nature of this technology allows for repeated monitoring, offering a safe and accessible method for early lymphoma screening and diagnosis. It also holds promise in tracking disease progression, enabling clinicians to make timely interventions.

### SUMMARY OF THE INVENTION

This invention provides a portable hyperspectral imaging device based on a CMOS spectral sensor chip, capable of collecting multispectral data through multi-wavelength light emitted by natural light or halogen lamp sources, and analyzing the spectral characteristics of the eyes, oral cavity, and skin in real-time. The device utilizes a high-resolution, high-sensitivity CMOS spectral sensor to capture spectral features from various critical components in human tissues and, through proprietary algorithms, analyze the concentrations of glucose, bilirubin, oxyhemoglobin (HbO2), and deoxyhemoglobin (Hb), thereby achieving early diagnosis of diseases such as diabetes, liver cancer, pancreatic cancer, and lymphoma. The device primarily detects corresponding conditions based on spectral curves, spectral images, and spectral models, while also referencing the sensitive wavelengths of glucose, bilirubin, and other substances.

Key advantages include:
High Flexibility: The compact design integrates a multispectral sensor and adjustable light source, enabling automatic adaptation to different detection environments. Users can easily collect and analyze spectral data through simple operation, making the device suitable for non-professional users and applications in hospitals, clinics, home care, and telemedicine.
Real-time Data Analysis: The device integrates a real-time data processing module that quickly calculates changes in glucose, bilirubin, HbO2, and Hb concentrations from spectral data. Through a user-friendly interface, it provides immediate feedback. The device can connect to smart devices, supporting wireless data transmission and remote monitoring, allowing doctors and patients to track health conditions in real time.
Low-cost, Portable Design: By using low-cost CMOS spectral sensors and a modular design, the manufacturing and maintenance costs of the device are reduced, making it widely applicable in various medical settings, including resource-constrained regions. The device's small form factor also makes it easy to carry and operate, meeting the needs of mobile healthcare.
Scalability and Compatibility: The device supports integration with other medical systems, enabling seamless data interfaces with electronic health records (EHRs) and health monitoring platforms. In the future, its detection capabilities can be enhanced through software upgrades and modular extensions, increasing the variety of detectable metabolic indicators and diseases.

### BRIEF DESCRIPTION OF THE DRAWINGS

FIG. 1 is a flowchart of the detection method based on the eyes, oral cavity, and/or skin.

FIG. 2 is a schematic diagram of the structure of the detection device for the eyes, oral cavity, and/or skin.

FIG. 3 is a schematic diagram of the structure of the mobile terminal.

### DETAILED DESCRIPTION OF THE INVENTION

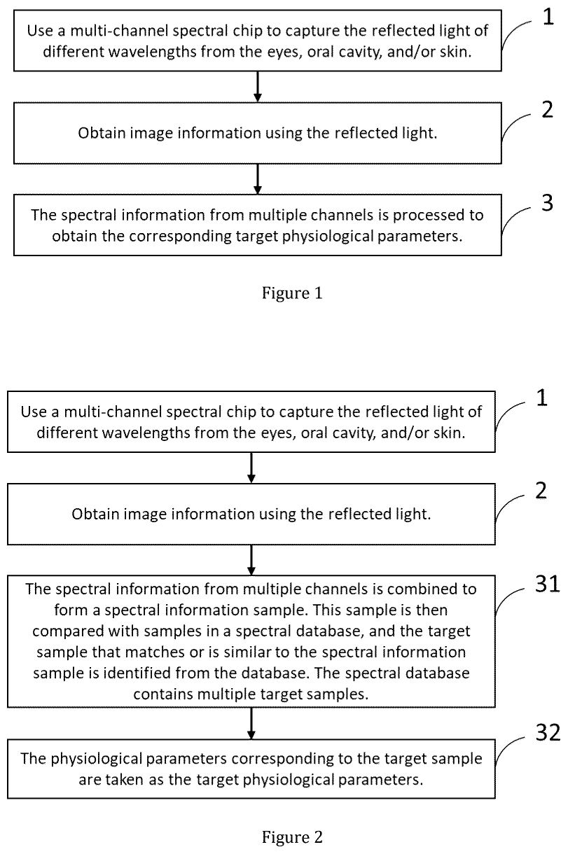

FIG. 1 shows flowchart six of the detection method based on the eyes, oral cavity, and/or skin. The method includes filtering the light based on its spectral information to obtain the corresponding spectral information to be tested from the eyes, oral cavity, and/or skin, and using this spectral information as the initial data, include:
Step 1. Emitting light source signals toward the eyes, oral cavity, and/or skin, with the light source signals comprising light of wavelengths corresponding to each channel. When indoor lighting is sufficient, natural light can be used as the light source signal without needing additional lighting. In dim indoor environments, a halogen lamp can be used as the light source, as the light from halogen lamps is relatively bright and focused, which helps capture the reflection of different wavelengths from the sclera (white of the eyes, oral cavity, and/or skin).
Step 2. Using a multispectral chip to capture the light reflected from the sclera of the eyes, oral cavity, and/or skin at different wavelengths.
Step 3. Obtaining image information using the reflected light.
Step 4. Determining the test areas corresponding to the eyes, oral cavity, and/or skin from the image information.
Step 5. Acquiring the spectral information corresponding to the test areas to obtain the spectral information to be tested from the eyes, oral cavity, and/or skin, and using this information as the initial data. In some embodiments, the test areas of the eyes, oral cavity, and/or skin can be determined from the image information by setting a coordinate system to roughly locate the areas. Specific regions or multiple regions of the image information can be selected as test areas, and the corresponding spectral information can be obtained to acquire the spectral data to be tested from the eyes, oral cavity, and/or skin.
Step 6. Processing the initial data to obtain spectral information for each channel, where different channels correspond to spectral information at different wavelengths.
Step 7. Processing the spectral information from multiple channels to derive the corresponding target physiological parameters.

It can be understood that natural light includes visible light and non-visible light, where visible light and non-visible light correspond to different preset wavelength ranges. Visible spectra include seven main colors, while non-visible light refers to light that the human eye cannot see, which extends beyond the visible spectrum to cover a wider wavelength range, including near-infrared (NIR), mid-infrared (MIR), and others.

FIG. 2 shows a schematic diagram of the structure of the detection device for the eyes, oral cavity, and/or skin. This application also provides a detection device 8 for the eyes, oral cavity, and/or skin, which is applied to a mobile terminal 11. The detection device 8 includes a multispectral chip 9 and a processor 10. The multispectral chip 9 is used to capture the light reflected from the sclera of the eyes, oral cavity, and/or skin at different wavelengths to obtain initial data. The processor 10 is used to process the initial data to derive the spectral information for each channel, where different channels correspond to spectral information at different wavelengths. The processor is also used to process the spectral information from multiple channels to derive the corresponding target physiological parameters. Additionally, a computer-readable storage medium is provided, on which a computer program is stored. When executed by the processor, the computer program implements the steps of the control method described in the claims.

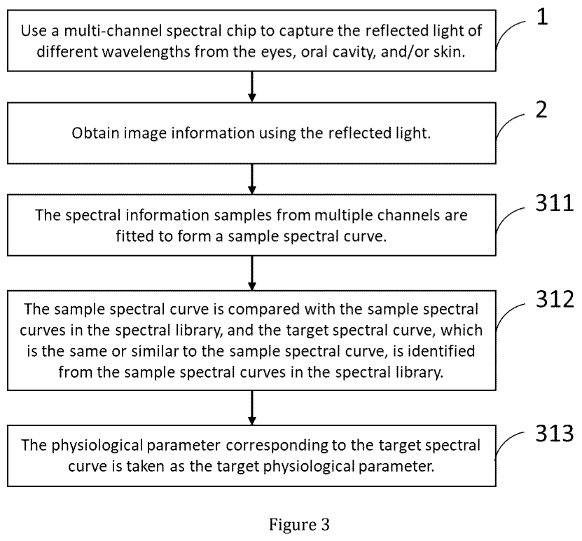

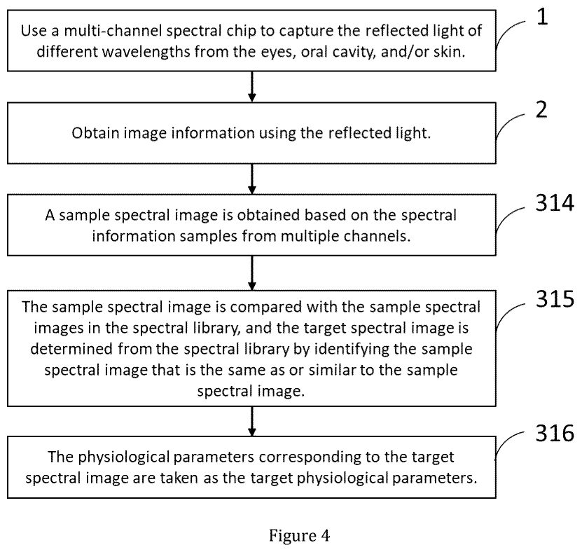

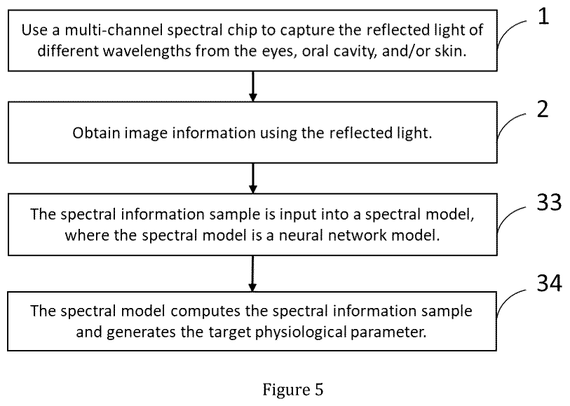

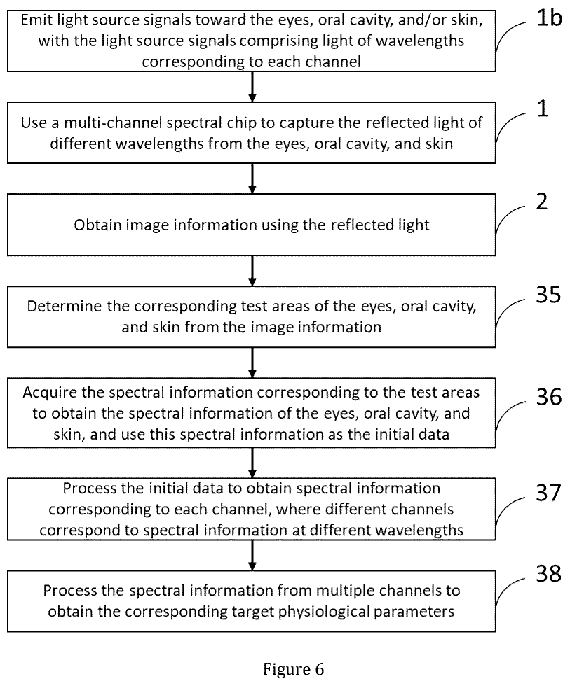

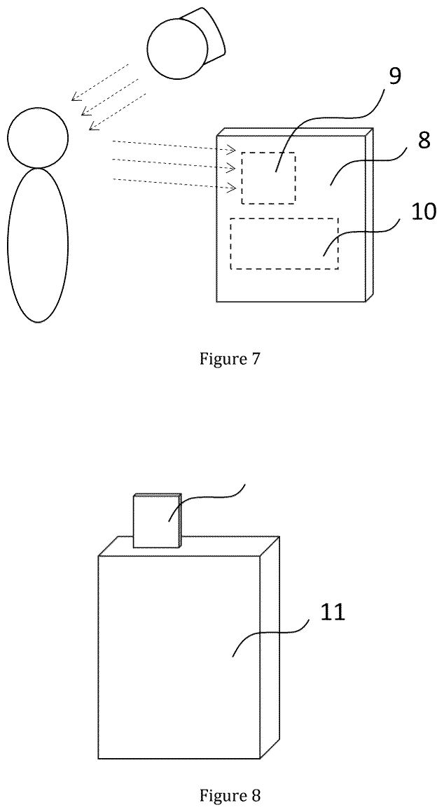

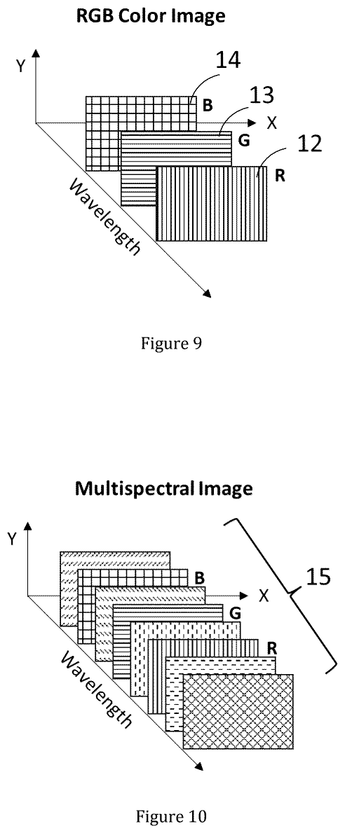

FIG. 3 shows a schematic diagram of the structure of the mobile terminal. This application also provides a mobile terminal 11, which includes the detection device 8, a memory, and a computer program stored in the memory that can be run on the processor. When executed by the processor, the computer program implements the steps of the control method described in the claims. In some embodiments, the mobile terminal 11 also includes a camera, with the detection device 8 arranged adjacent to the camera, which can consist of one or more cameras. In some embodiments, the mobile terminal 11 includes a display screen, which is connected to the detection device 8 and can display the target physiological parameters.

1. A detection method based on the eyes, oral cavity, and/or skin, comprising:
Utilizing a multispectral chip to acquire light of different wavelengths reflected from the eyes, oral cavity, and/or skin to obtain initial information;
Processing the initial information to obtain spectral information corresponding to each channel, wherein different channels correspond to spectral information of different wavelengths;
Processing the spectral information from multiple channels to obtain corresponding target physiological parameters.

2. The detection method based on the eyes, oral cavity, and/or skin according to claim 1, further comprising, before utilizing the multispectral chip to acquire light of different wavelengths reflected from the eyes, oral cavity, and/or skin to obtain initial information:
Emitting a light source signal to the eyes, oral cavity, and/or skin, wherein the light source signal comprises light of wavelengths corresponding to each channel.

3. The detection method based on the eyes, oral cavity, and/or skin according to claim 1, wherein utilizing the multispectral chip to acquire light of different wavelengths reflected from the eyes, oral cavity, and/or skin to obtain initial information includes:
Utilizing a multispectral chip to acquire light of different wavelengths reflected from the eyes, oral cavity, and/or skin;
Filtering the acquired light based on spectral information to obtain the corresponding spectral information of the eyes, oral cavity, and/or skin to be tested, and using the obtained spectral information as the initial information.

4. The detection method based on the eyes, oral cavity, and/or skin according to claim 3, wherein filtering the acquired light based on spectral information to obtain the corresponding spectral information of the eyes, oral cavity, and/or skin to be tested, and using the obtained spectral information as the initial information includes:
Using the acquired light to obtain image information;
Determining the test area corresponding to the eyes, oral cavity, and/or skin from the image information;
Acquiring spectral information corresponding to the test area to obtain the spectral information of the eyes, oral cavity, and/or skin to be tested, and using the obtained spectral information as the initial information.

5. The detection method based on the eyes, oral cavity, and/or skin according to claim 1, wherein the target physiological parameters include glucose, bilirubin, oxyhemoglobin (HbO2) and/or deoxyhemoglobin (Hb) parameters.

6. An eyes, oral cavity, and/or skin detection device, applied in a mobile terminal, characterized in that the eyes, oral cavity, and/or skin detection device comprises:
A multispectral chip for acquiring light of different wavelengths reflected from the eyes, oral cavity, and/or skin to obtain initial information;
A processor for processing the initial information to obtain spectral information corresponding to each channel, wherein different channels correspond to spectral information of different wavelengths; the processor is further used to process the spectral information from multiple channels to obtain the corresponding target physiological parameters.

7. A computer-readable storage medium, characterized in that a computer program is stored on the computer-readable storage medium, which, when executed by a processor, implements the steps of the control method according to any one of claims 1-5.

8. A mobile terminal, characterized in that it comprises a processor, a memory, and a computer program stored in the memory and executable on the processor, wherein the computer program, when executed by the processor, implements the steps of the control method according to any one of claims 1-5.
